(19) United States
(12) Patent Application Publication
(10) Pub. No.: US 2010/0137899 A1
(43) Pub. Date: Jun. 3, 2010

(54) Mechanical Embolectomy Device and Method

(76) Inventor: Nasser Razack, Bradenton, FL (US)

Correspondence Address: Maxey Law Offices, PLLC, 13630 58TH ST. NORTH, SUITE 101, CLEARWATER, FL, 33760, US

(21) Appl. No.: 12/326,459

(22) Filed: Dec. 2, 2008

(51) Int. Cl. A61M 29/02

(52) U.S. Cl. 606/200

(57) ABSTRACT

An embolectomy device includes a proximal elongated shaft positionable and movable within a catheter, the proximal elongated shaft having a distal end. A distal elongated shaft positionable and movable with a catheter, the distal elongated shaft having a proximal end. An expander portion comprising a body having proximal and distal ends. The proximal end of the body extending from the distal end of the proximal elongated shaft and the proximal end of the distal elongated shaft extending from the distal end of the body of the expander portion. The body being biased to expand laterally outward from an axis of the embolectomy device. The body is shaped as an spheroid when expanded laterally outward.

### FILED OF THE INVENTION

The present invention relates generally to embolectomy, and more particularly, relating to a mechanical embolectomy device including a clot expander, and method of using the same.

### BACKGROUND OF THE INVENTION

There are currently in excess of 700,000 new or recurrent strokes every year in the United States. It is the third leading cause of death in the United States after coronary artery disease and cancer. Approximately 40% (>250,000) of strokes are due to large vessel occlusion, potentially requiring a device for treatment.

Treatment must begin with an evaluation of the patient. Diagnostic neuroimaging is used to obtain noninvasive real-time information about the patient. The goal of therapy is to restore perfusion to the ischemic but potentially salvageable brain tissue rather than to the irreversibly damaged brain tissue, since re-establishing blood flow to such damaged tissue can cause complications such as hemorrhage. Determining the cause and location of the blockage is critical to planning the treatment approach. The most common cause of ischemic stroke is acute embolic occlusion. Most patients with acute ischemic stroke have thromboembolic material occluding large cerebral vessels and hence disruption of cerebral blood flow. Removal of the arterial occlusion in a timely manner can provide a substantial reduction in the size and severity of the cerebral infarction, and improvement in the level of disability among survivors. Treatment varies depending on whether the lesion is proximal or distal, whether there is underlying atherosclerotic stenosis at the occlusion site, and whether the proximal extracranial vessel is opened or closed. Where there are proximal arterial occlusions, the physician may attempt clot retrieval, supplemented by direct catheter-directed thrombolysis. Severe stenosis proximal to the occlusion will usually require treatment of the stenosois before or immediately after restoring intracranial flow.

A current treatment for acute ischemic stroke is intravenous thrombolysis using tissue-type plasminogen activator (TPA). TPA is a naturally occurring enzyme that activates plasminogen into active plasmin, which dissolves fibrin. The dissolution of fibrin in a clot causes thrombolysis. This treatment is suitable for smaller clots, but has limited utility for patients with large clots, such as are often present in acute occlusions of the internal carotid artery (ICA), proximal middle cerebral artery (MCA), and basilar artery (BA). Also, TPA therapy has significant time constraints, and is generally effective only if given within 3-6 hours of stroke symptom onset. Contraindications to TPA and these time constraints led to mechanical embolectomy.

Mechanical removal of the thrombus is the goal of mechanical embolectomy. Lytic therapy is necessary for non-accessible locations. Mechanical embolectomy is the process by which a mechanical device is inserted into the body, moved through the affected body canal to the site of the occlusion, and then used to mechanically remove the occlusion from the canal to restore blood flow. One such device is the Mechanical Embolus Removal in Cerebral Ischemia (Merci) retrieval device (Concentric Medical, Mountain View, Calif.), which is currently available for routine clinical use in acute ischemic stroke within 9 hours of onset. This device is a flexible and tapered nickel titanium wire with a helically shaped distal tip that can be deployed intra-arterially to entrap and retrieve large vessel intracerebral clots. Other devices are in various stages of development.

### SUMMARY OF THE INVENTION

In general, in one aspect, an embolectomy device is provided. The embolectomy device includes a proximal elongated shaft positionable and movable within a catheter, the proximal elongated shaft having a distal end. A distal elongated shaft positionable and movable with a catheter, the distal elongated shaft having a proximal end. An expander portion comprising a body having proximal and distal ends. The proximal end of the body extending from the distal end of the proximal elongated shaft and the proximal end of the distal elongated shaft extending from the distal end of the body of the expander portion. The body being biased to expand laterally outward from an axis of the embolectomy device. The body is shaped as an spheroid when expanded laterally outward.

The proximal end of the body of the expander portion can extend proximally along the proximal elongated shaft, and the distal end of the body of the expander portion can extend distally along the distal elongated shaft. A first band can secure the proximal end of the body of the expander portion to the proximal elongated shaft, and a second band can secure the distal end of the body of the expander portion to the distal elongated shaft. The first band and the second band can be radio opaque. A pair of radio opaque markers, can located at each of the proximal end and the distal end of the expander portion. The body of the expander portion can be elongated pointed prolate spheroid. The body of the expander portion can be prolate spheroid.

In general, in another aspect, an embolectomy device is provided. The embolectomy device includes an elongated shaft positionable and movable within a catheter. The elongated shaft having a distal end. An expander portion comprising a body having proximal and distal ends. The elongated shaft extending through the body of the expander portion with the proximal end and the distal end of the body of the expander portion being attached to the elongated shaft at a spaced distance along the elongated shaft. The body of the expander portion being biased to expand laterally outward from an axis of the embolectomy device. The body of the expander portion is shaped as an spheroid when expanded laterally outward.

The proximal end of the body of the expander portion can extend proximally along the elongated shaft, and the distal end of the body of the expander portion can extend distally along the elongated shaft. A first band can secure the proximal end of the body of the expander portion to the elongated shaft. A second band can secure the distal end of the body of the expander portion to the elongated shaft. The first band and the second band can be radio opaque. A pair of radio opaque markers, can be located one at each of the proximal end and the distal end of the expander portion. The body of the expander portion can be elongated pointed prolate spheroid. The body of the expander portion can be prolate spheroid.

There has thus been outlined, rather broadly, the more important features of the invention in order that the detailed description thereof that follows may be better understood and in order that the present contribution to the art may be better appreciated.

Numerous objects, features and advantages of the present invention will be readily apparent to those of ordinary skill in the art upon a reading of the following detailed description of presently preferred, but nonetheless illustrative, embodiments of the present invention when taken in conjunction with the accompanying drawings. The invention is capable of other embodiments and of being practiced and carried out in various ways. Also, it is to be understood that the phraseology and terminology employed herein are for the purpose of descriptions and should not be regarded as limiting.

As such, those skilled in the art will appreciate that the conception, upon which this disclosure is based, may readily be utilized as a basis for the designing of other structures, methods and systems for carrying out the several purposes of the present invention. It is important, therefore, that the claims be regarded as including such equivalent constructions insofar as they do not depart from the spirit and scope of the present invention.

For a better understanding of the invention, its operating advantages and the specific objects attained by its uses, reference should be had to the accompanying drawings and descriptive matter in which there is illustrated preferred embodiments of the invention.

### BRIEF DESCRIPTION OF THE DRAWINGS

The accompanying drawings, which are included to provide further understanding of the invention and are incorporated in and constitute a part of this specification, illustrate preferred embodiments of the invention and together with the description serve to explain the principles of the invention, in which:

FIG. 1 is a side elevation view of an embolectomy device in accordance with the principles of the present invention;

FIG. 2 is an enlarge perspective view of the embolectomy device of FIG. 1;

FIG. 3 is a longitudinal cross-section of the embolectomy device of FIG. 1;

FIGS. 4-7 are side elevation views of the embolectomy device of FIG. 1 shown at various stages of operation spread clot material concentrically outwardly towards the walls of the body canal, opening a lumen through the body canal;

FIG. 8 is a side elevation view an alternate embodiment of an embolectomy device in accordance with the principles of the present invention; and

FIG. 9 is a longitudinal cross-section of the embolectomy device of FIG. 8.

### DETAILED DESCRIPTION OF THE INVENTION

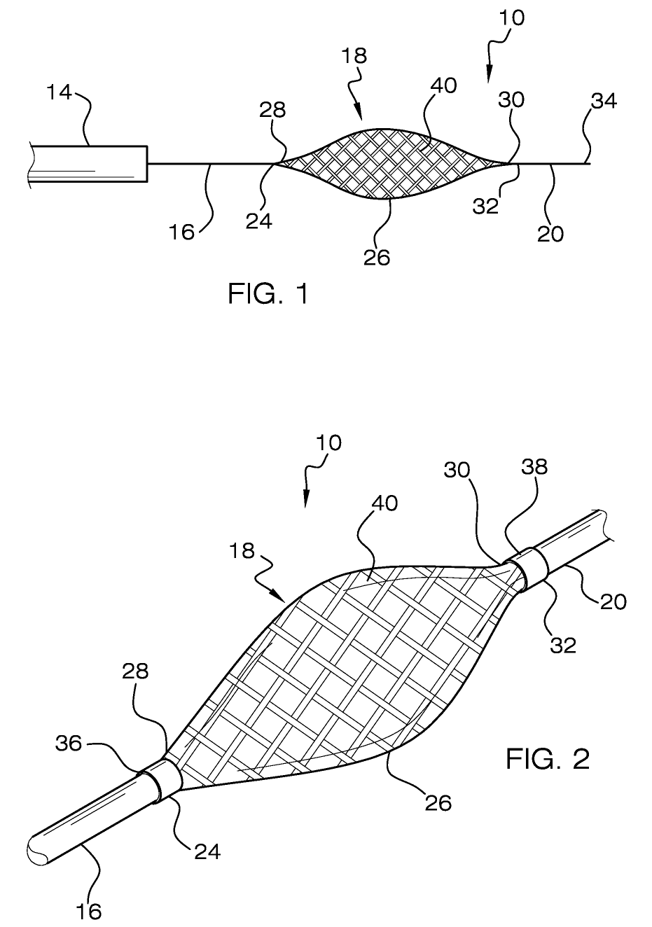

In FIGS. 1-2, there is shown an embolectomy device 10 according to the invention. The embolectomy device 10 is positionable in and movable within a catheter, such as microcatheter 14. The embolectomy device 10 includes a proximal elongated shaft such as proximal wire 16, an expander portion 18, and a distal elongated shaft such as distal wire 20. The proximal wire 16 includes a proximal end (not shown) and a distal end 24. The expander portion 18 comprises a body 26 having proximal and distal ends 28 and 30, respectively. The distal wire 20 includes a proximal and distal ends 32 and 34, respectively.

The body 26 of the expander portion is disposed about the long axis A of the embolectomy device 10, and is biased or otherwise moveable to extend or expand laterally outward when not constrained within an outer sheath, such as microcatheter 14. When body 26 of the expander portion 18 is not constrained and is fully extended or expanded laterally outward, as shown in FIGS. 1 and 2, the body has a spheroid-like shape. A spheroid is a quadric surface obtained by rotating an ellipse about one of its principal axes; in other words, an ellipsoid with two equal semi-diameters. Preferably, body 34 is a prolate spheroid, which is formed if the ellipse is rotated about its major axis. Most preferably, the body is an elongated pointed prolate spheroid, which is defined herein as a prolate spheroid that is elongated along its major axis such that opposite ends of the prolate spheroid on the major axis are elongated in opposite outwardly directions resulting in a prolate spheroid body that tapers conically at both ends. The body 26 of the expander portion 18 may be symmetrical or may be asymmetrical about both or either of its major and minor axis.

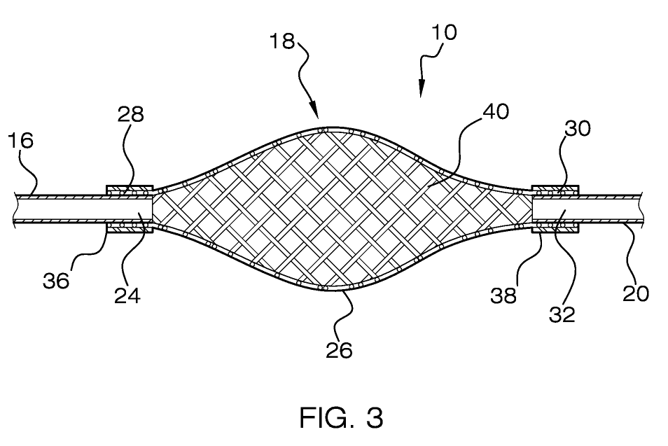

The body 26 of the expander portion 18 may be formed with the proximal and distal wires 16 and 20, respectively. Proximal and distal ends 28 and 30 of the body 26 may be are attached to proximal and distal wires 16 and 20, respectively, for example by such methods as welding, clamping, bonding, polymer encapsulation or by other suitable methods. As shown in FIG. 3, proximal end 28 of the body 26 may extend in a direction proximally along proximal wire 16, and secured in place by a circular band 36 that is crimped or otherwise compressed about the proximal end 28 and the distal end 24 of the proximal wire. Likewise, distal end 30 of the body 26 may extend in a direction distally along distal wire 20, and secured in place by a circular band 38 that is crimped or otherwise compressed about distal end 30 and proximal end 32 of the distal wire. Circular bands 36 and 38 can be replaced by or used in addition to welding, bonding, polymer encapsulation or other suitable attachment means or method. Circular bands 36 and 38 may be radio opaque markers. Other forms of radio opaque markers may be included at the proximal and distal ends 30 and 32 of the body 26 of the expander portion 16.

The expander portion 18 comprising body 26 can be of different designs, but can be a mesh metallic or polymeric device similar to a stent that is capable of a first position in which it is tightly compacted laterally, and in another position expands laterally. It is important to understand the expander portion 18 is only stent-like, and it is not intended to be left in place within a body canal. The expander portion 18 use this later expansion force to spread clot material concentrically outwardly towards the walls of the body canal, opening a lumen through the body canal. In the high energy state, compacted state the expander portion 18 is contained within a sheath cover, such as a catheter. When the expander portion 18 is not contained within the catheter, the expander portion assumes an expanded, lower energy state.

The expander portion 18 can be completely or partially coated with a polymer, and/or with a therapeutic substance. The expander portion 18 can be porous, non-porous, or partially porous. The expander portion 18 can be can be formed by suitable techniques, such as, but not limited to, computer-controlled laser cutting, plastic injection molding, or casting.

A mesh or net 40 can cover a portion or all of the expander portion 16 in order to better engage clot material. The net 40 can be constructed from a porous or a substantially non-porous material, such as a flexible plastic net or solid sheet material, and/or a biocompatible or non-thrombogenic polymer. The net 40 can be integral with the expander portion.

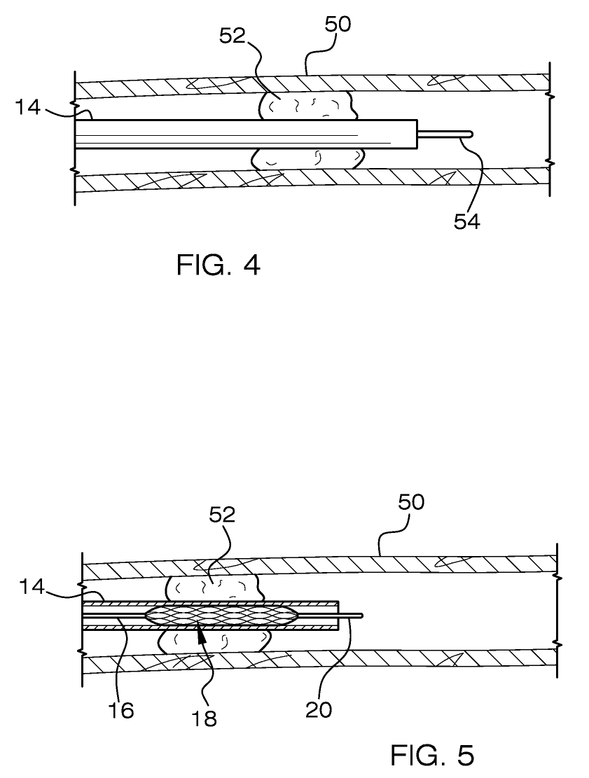

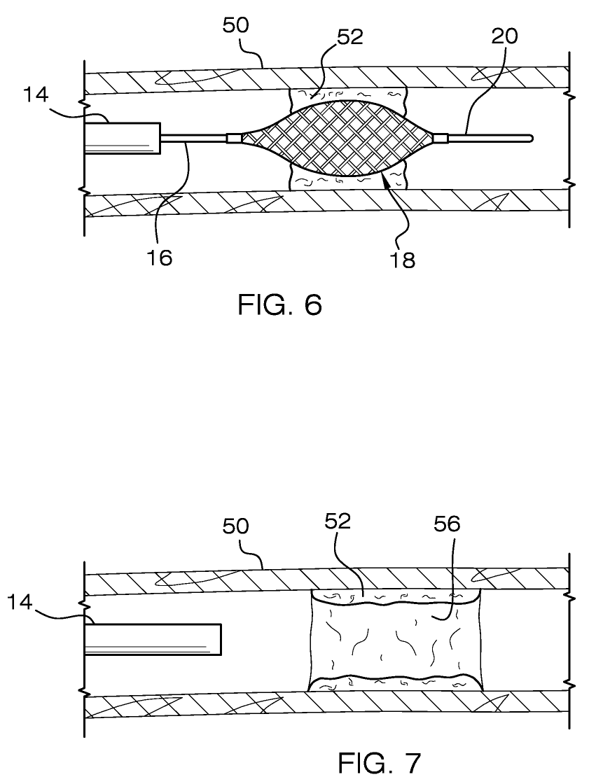

Operation of the embolectomy device 10 is shown in FIGS. 4-7. In FIG. 4, the microcatheter 14 is shown positioned within the body canal 50 through and beyond clot 52 using a steerable microwire 54 and standard interventional radiology techniques. With the microcatheter 14 in position in the body canal 50 and through the clot 52, the steerable microwire is removed from the microcatheter. A contrasting agent can be injected into the microcatheter 14 to confirm the microcatheter is correctly positioned through the clot 52. While maintaining the position of microcatheter 14 the embolectomy device 10 is inserted into the microcatheter to position the expander portion 18 across the clot 52 (FIG. 5). The embolectomy device 10 is extended from the microcatheter 14 by withdrawing the microcatheter 14 proximally while maintaining the positioning of the embolectomy device. Continued withdrawal of the microcatheter 14 exposes the expander portion 18, which is expands, pressing the clot 52 against the body canal 50 (FIG. 6) and creating a lumen. The expander portion 18 can then be collapsed and retracted into the microcatheter 14 by moving the microcatheter distally over the expander portion. With the expander portion 18 contained within the microcatheter 14, the embolectomy device 10 can be retracted leaving the lumen 56 and at least partially restoring fluid flow through the body canal 50 (FIG. 7).

The dimensions and construction of the microcatheter 14, elongated shaft or proximal wire 16, expander portion 18, and distal wire 20 can vary depending on the size of the canal in which the clot is located, the size and position of the clot, and other factors. The dimensions of the proximal and distal wires 16 and 20 can in one embodiment be 0.35 mm in diameter, and between 0.20 mm to 0.45 mm in diameter. In another embodiment, the dimensions of the proximal and distal wires 16 and 20 can be 0.25 mm, and between 0.20 mm to 0.36 mm. The dimensions of the microcatheter 14 can be an outside diameter (OD) 0.60 mm, and an inside diameter (ID) of 0.43 mm, or with an (OD) between 0.40 mm to 1.37 mm, and an (ID) between 0.25 mm to 0.75 mm. The dimensions of the expander portion 18 can, for example, be between 10 mm and 44 mm in length. Other dimensions are possible.

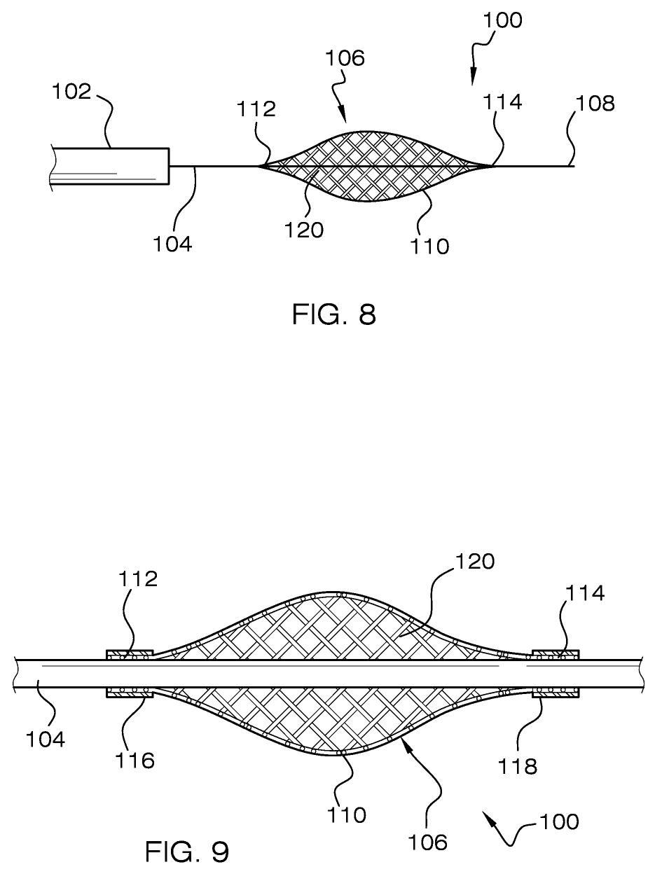

Other embodiments are possible. In FIG. 8, there is shown a longitudinal cross-section of a second embodiment of an embolectomy device 100. The embolectomy device 100 is positionable in and movable within a catheter, such as microcatheter 102. In this embodiment, the embolectomy device 100 includes an elongated shaft or wire 104 and an expander portion 106. The wire 104 includes a proximal end (not shown) and a distal end 108. The expander portion 106 comprises a body 110 having proximal and distal ends 112 and 114, respectively. Wire 104 extends through the body 1 10 of the expander portion 106.

The body 110 of the expander portion 106 is disposed about the long axis A of the embolectomy device 100, and is biased or otherwise moveable to extend or expand laterally outward when not constrained within an outer sheath, such as microcatheter 102. When body 1 10 of the expander portion 106 is not constrained and is fully extended or expanded laterally outward, as shown in FIGS. 8 and 9, the body has a spheroid-like shape. A spheroid is a quadric surface obtained by rotating an ellipse about one of its principal axes; in other words, an ellipsoid with two equal semi-diameters. Preferably, body 110 is a prolate spheroid, which is formed if the ellipse is rotated about its major axis. Most preferably, the body is an elongated pointed prolate spheroid, which is defined herein as a prolate spheroid that is elongated along its major axis such that opposite ends of the prolate spheroid on the major axis are elongated in opposite outwardly directions resulting in a prolate spheroid body that tapers conically at both ends.

The body 110 of the expander portion 106 may be formed with wire 104. Proximal and distal ends 28 and 30 of the body 110 may be attached to wire 104 at spaced distances along wire, for example by such methods as welding, clamping, bonding, polymer encapsulation or by other suitable methods. As shown in FIG. 9, proximal end 112 of the body 110 may extend in a direction proximally along proximal wire 104, and secured in place by a circular band 116 that is crimped or otherwise compressed about the proximal end 112 and wire 104. Likewise, distal end 114 of the body 110 may extend in a direction distally along wire 104, and secured in place by a circular band 118 that is crimped or otherwise compressed about distal end 114 and wire 104. Circular bands 116 and 118 can be replaced by or used in addition to welding, bonding, polymer encapsulation or other suitable attachment means or method. Circular bands 116 and 118 may be radio opaque markers. Other forms of radio opaque markers may be included at the proximal and distal ends 112 and 114 of the body 110 of the expander portion 106.

The expander portion 106 comprising body 110 can be of different designs, but can be a mesh metallic or polymeric device similar to a stent that is capable of a first position in which it is tightly compacted laterally, and in another position expands laterally. It is important to understand the expander portion 106 is only stent-like, and it is not intended to be left in place within a body canal. The expander portion 106 use this later expansion force to spread clot material concentrically outwardly towards the walls of the body canal, opening a lumen through the body canal. In the high energy state, compacted state the expander portion 106 is contained within a sheath cover, such as a catheter. When the expander portion 106 is not contained within the catheter, the expander portion assumes an expanded, lower energy state.

The expander portion 106 can be completely or partially coated with a polymer, and/or with a therapeutic substance. The expander portion 106 can be porous, non-porous, or partially porous. The expander portion 106 can be can be formed by suitable techniques, such as, but not limited to, computer-controlled laser cutting, plastic injection molding, or casting.

A mesh or net 120 can cover a portion or all of the expander portion 106 in order to better engage clot material. The net 120 can be constructed from a porous or a substantially non-porous material, such as a flexible plastic net or solid sheet material, and/or a biocompatible or non-thrombogenic polymer. The net 120 can be integral with the expander portion.

Operation of the embolectomy device 100 would be the same as the operation of embolectomy device 10 as discussed above and shown in FIGS. 4-7.

The dimensions and construction of the microcatheter 102, elongated shaft or wire 104 and expander portion 106 can vary depending on the size of the canal in which the clot is located, the size and position of the clot, and other factors. The dimensions of the wire 104 can in one embodiment be 0.35 mm in diameter, and between 0.20 mm to 0.45 mm in diameter. In another embodiment, the dimensions of the wire 104 can be 0.25 mm, and between 0.20 mm to 0.36 mm. The dimensions of the microcatheter 102 can be an outside diameter (OD) 0.60 mm, and an inside diameter (ID) of 0.43 mm, or with an (OD) between 0.40 mm to 1.37 mm, and an (ID) between 0.25 mm to 0.75 mm. The dimensions of the expander portion 106 can, for example, be between 10 mm and 44 mm in length. Other dimensions are possible.

A number of embodiments of the present invention have been described. Nevertheless, it will be understood that various modifications may be made without departing from the spirit and scope of the invention. Accordingly, other embodiments are within the scope of the following claims.

What is claimed is:

1. An embolectomy device, comprising:
a proximal elongated shaft positionable and movable within a catheter, said proximal elongated shaft having a distal end;
a distal elongated shaft positionable and movable with a catheter, said distal elongated shaft having a proximal end;
an expander portion comprising a body having proximal and distal ends, said proximal end of said body extending from said distal end of said proximal elongated shaft and said proximal end of said distal elongated shaft extending from said distal end of said body of said expander portion;
wherein said body being biased to expand laterally outward from an axis of the embolectomy device; and
wherein said body is shaped as an spheroid when expanded laterally outward.

2. The embolectomy device of claim 1, wherein:
said proximal end of said body of said expander portion extends proximally along said proximal elongated shaft; and
said distal end of said body of said expander portion extends distally along said distal elongated shaft.

3. The embolectomy device of claim 2, further comprising:
a first band securing said proximal end of said body of said expander portion to said proximal elongated shaft; and
a second band securing said distal end of said body of said expander portion to said distal elongated shaft.

4. The embolectomy device of claim 3, wherein:
said first band and said second band are radio opaque.

5. The embolectomy device of claim 1, further comprising:
a pair of radio opaque markers, one located at each of said proximal end and said distal end of said expander portion.

7. The embolectomy device of claim 1, wherein said body of said expander portion is an elongated pointed prolate spheroid.

8. The embolectomy device of claim 1, wherein said body of said expander portion is a prolate spheroid.

9. An embolectomy device, comprising:
an elongated shaft positionable and movable within a catheter, said elongated shaft having a distal end;
an expander portion comprising a body having proximal and distal ends;
said elongated shaft extending through said body of said expander portion with said proximal end and said distal end of said body of said expander portion being attached to said elongated shaft at a spaced distance along said elongated shaft;
wherein said body being biased to expand laterally outward from an axis of the embolectomy device; and
wherein said body is shaped as an spheroid when expanded laterally outward.

10. The embolectomy device of claim 9, wherein:
said proximal end of said body of said expander portion extends proximally along said elongated shaft; and
said distal end of said body of said expander portion extends distally along said elongated shaft.

11. The embolectomy device of claim 10, further comprising:
a first band securing said proximal end of said body of said expander portion to said elongated shaft; and
a second band securing said distal end of said body of said expander portion to said elongated shaft.

12. The embolectomy device of claim 11, wherein:
said first band and said second band are radio opaque.

13. The embolectomy device of claim 9, further comprising:
a pair of radio opaque markers, one located at each of said proximal end and said distal end of said expander portion.

14. The embolectomy device of claim 9, wherein said body of said expander portion is an elongated pointed prolate spheroid.

15. The embolectomy device of claim 9, wherein said body of said expander portion is a prolate spheroid.

16. A method of performing an embolectomy, comprising the steps of:
providing an embolectomy device comprising a proximal elongated shaft positionable and movable within a catheter, said proximal elongated shaft having a distal end, a distal elongated shaft positionable and movable with a catheter, said distal elongated shaft having a proximal end, an expander portion comprising a body having proximal and distal ends, said proximal end of said body extending from said distal end of said proximal elongated shaft and said proximal end of said distal elongated shaft extending from said distal end of said body of said expander portion, wherein said body being biased to expand laterally outward from an axis of the embolectomy device, and wherein said body is shaped as an spheroid when expanded laterally outward manipulating the embolectomy device within a catheter to position the body across an occlusion in a body canal;
expanding the body to move the occlusion toward the body canal, thereby creating a lumen within the canal;
retracting the body; and
removing the catheter and expander portion from the body canal leaving the lumen open.
